(19) United States
(12) Patent Application Publication
(10) Pub. No.: US 2014/0194528 A1
(43) Pub. Date: Jul. 10, 2014

(54) USAGE OF SODIUM HOUTTUYFONATE ON INHIBITING IDIOPATHIC PULMONARY FIBROSIS AND BLEOMYCIN INDUCED PULMONARY FIBROSIS

(71) Applicant: Hong Kong Baptist University, Hong Kong (HK)

(72) Inventors: Hongxi XU, HONG KONG (HK); Yunhui SHEN, HONG KONG (HK); Yuanzhi LAO, HONG KONG (HK); Kaixian CHEN, HONG KONG (HK); Dajian YANG, HONG KONG (HK); Shilin CHEN, HONG KONG (HK); Zhaoxiang BIAN, HONG KONG (HK); Aiping LU, HONG KONG (HK); Albert Sun Chi CHAN, HONG KONG (HK); Chengyuan LIN, HONG KONG (HK)

(73) Assignee: Hong Kong Baptist University, Hong Kong (HK)

(21) Appl. No.: 14/149,823

(22) Filed: Jan. 8, 2014

Related U.S. Application Data
(60) Provisional application No. 61/750,859, filed on Jan. 10, 2013.

(51) Int. Cl. A61K 31/121

(52) U.S. Cl. CPC A61K 31/121
USPC 514/675; 568/31

(57) ABSTRACT

The present invention relates to a synthetic compound and its therapeutic uses. More particularly, it relates to a compound, Sodium Houttuyfonate, that is an addition compound of sodium bisulfite and houttuynin, and its biological activity of inhibiting idiopathic pulmonary fibrosis and bleomycin induced pulmonary fibrosis

### CROSS-REFERENCE TO RELATED APPLICATIONS

This application claims the benefit of U.S. Provisional Patent Application Ser. No. 61/750,859 filed on Jan. 10, 2013, which is hereby incorporated by reference in its entirety.

### FIELD OF INVENTION

The present invention relates to a synthetic compound and its therapeutic uses. More particularly, it relates to a synthetic compound, Sodium Houttuyfonate (SH), that is an addition compound of sodium bisulfite and houttuynin, and its biological activity of inhibiting idiopathic pulmonary fibrosis and bleomycin induced pulmonary fibrosis.

### BACKGROUND OF INVENTION

Pulmonary fibrosis is a disease that involves abnormal wound healing and is characterized by lung destruction and dysfunction. The mechanisms responsible for the progression of lung fibrosis are complex and, besides fibroblast activation, include alterations in the immune response and its regulation and chronic inflammation. In addition to the well-established role of stimulatory pathways, the importance of natural counter-regulatory mechanisms, which are responsible for the preservation of tissue function through the limitation of inflammatory and fibrotic responses, is being recognized. There is growing evidence such as in Wilson M S, Wynn T A. Pulmonary fibrosis: pathogenesis, etiology and regulation. Mucosal Immunol 2009; 2(2): 103-21 that chronic inflammation results from insufficient production of anti-inflammatory and pro-resolving mediators, whereas fibrosis results from inadequate generation of the suppressive signals that control fibroblast function. Corticosteroids and other immunosuppressive agents have been used to treat pulmonary fibrosis, but their efficacy has been disappointing and new insights into the pathophysiology and the establishment of a new therapy are urgently needed.

Pulmonary fibrosis (PF) can severely disrupt lung function with fatal consequences. However, there is currently no effective treatment for PF and idiopathic pulmonary fibrosis (IPF). Therefore, there is a need for a new drug to effectively treat PF and IPF.

Citation or identification of any reference in this section or any other section of this application shall not be construed as an admission that such reference is available as prior art for the present application.

### SUMMARY OF INVENTION

Accordingly, it is an object of the present invention to provide a compound, Sodium Houttuyfonate, that is an addition compound of sodium bisulfite and houttuynin for the treatment and prevention of idiopathic pulmonary fibrosis and bleomycin induced pulmonary fibrosis.

Accordingly, in the first aspect of the present invention there is provided a composition for treating or preventing pulmonary fibrosis in a subject in need thereof comprising at least one derivative compound from sodium bisulfite and houttuynin or their chemical equivalent.

Accordingly, in the first embodiment of the first aspect of the present invention the derivative compound comprises sodium houttuyfonate or its chemical equivalent.

Accordingly, in the second embodiment of the first aspect of the present invention the pulmonary fibrosis comprises idiopathic pulmonary fibrosis and bleomycin induced pulmonary fibrosis.

Accordingly, in the third embodiment of the first aspect of the present invention the derivative compound is used in preventing or attenuating pulmonary toxicity in subjects with bleomycin induced pulmonary fibrosis.

Accordingly, in the fourth embodiment of the first aspect of the present invention the derivative compound is administered orally to a subject in need thereof wherein the oral administration is at a dosage between 45 mg/kg bodily weight and 90 mg/kg bodily weight for at least once a day.

Accordingly, in the fifth embodiment of the first aspect of the present invention the derivative compound is used in preventing pulmonary toxicity in subjects administered with bleomycin as chemotherapeutic agent causing bleomycin induced pulmonary fibrosis.

Accordingly, in the second aspect of the present invention there is provided a method for treating or preventing pulmonary fibrosis in a subject in need thereof comprising the use of the composition according to the first aspect of the present invention.

Accordingly, in the first embodiment of the second aspect of the present invention the derivative compound comprises sodium houttuyfonate or its chemical equivalent.

Accordingly, in the second embodiment of the second aspect of the present invention the pulmonary fibrosis comprise idiopathic pulmonary fibrosis and bleomycin induced pulmonary fibrosis.

Accordingly, in the third embodiment of the second aspect of the present invention the derivative compound is used in preventing or attenuating pulmonary toxicity in subjects with bleomycin induced pulmonary fibrosis.

Accordingly, in the fourth embodiment of the second aspect of the present invention the derivative compound is administered orally to a subject in need thereof wherein the oral administration is at a dosage between 45 mg/kg bodily weight and 90 mg/kg bodily weight for at least once a day.

Accordingly, in the fifth embodiment of the second aspect of the present invention the derivative compound is used in preventing pulmonary toxicity in subjects administered with bleomycin as chemotherapeutic agent causing bleomycin induced pulmonary fibrosis.

Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the effect of SH on lung/body weight ratio in BLM mice. Each bar represents the mean±SD in each group (n=6˜10). #p<0.05, ##p<0.01 compared with Control group, *p<0.05, * *p<0.01 compared with BLM group, in the same time course.

FIG. 2 shows the lung tissue morphological changes in BLM and SH treated mice (H&E staining, ×200 magnification).

FIG. 3 shows the SH attenuated BLM-induced lung fibrosis.

FIG. 3A shows Sirius Red staining of each group in different time course. Images were adapted in Bright Field (BF) and Polarized light (Sirius Red, SR) in the same field (×200 magnification);

FIG. 3B shows collagen I and III. Ratio in each group in different time course. Ratio is calculated by measuring the intensity of Orange/yellow color (Collagen I) and green color (Collagen III). Samples are from more than three animals and statistic results are from more than 5 images in each sample;

FIG. 3C shows hydroxyproline contents in each group in different time course. Each bar represents the mean±SD in each group (n=6˜10). #p<0.05, ##p<0.01 compared with Control group, *p<0.05, * *p <0.01 compared with BLM group, in the same time course.

### DETAILED DESCRIPTION OF INVENTION

The present invention is not to be limited in scope by any of the specific embodiments described herein. The following embodiments are presented for exemplification only.

Sodium houttuyfonate (SH), an addition compound of sodium bisulfite and houttuynin, is shown to be effective on treating or attenuating idiopathic pulmonary fibrosis (IPF) or bleomycin (BLM) induced pulmonary fibrosis in the present invention. The experimental results provided hereinafter indicate that SH is capable to attenuate the syndrome of BLM induced lung injury, such as reducing the inflammation, lung fibrogenesis and the increase in lung/body weight ratio. The mechanisms underlying the protective effects of SH include impairment of the collagen formation which has been studied in vivo in BLM mice. Furthermore, the protective effects of SH against BLM induced pulmonary fibrosis in mice are comparable to that of prednisone acetate (PA) tablets, a widely used drug in immune disease. The experimental results provide the evidence that SH is an effective compound against Idiopathic Pulmonary Fibrosis and Bleomycin Induced Pulmonary Fibrosis.

Materials and Methods

Chemicals

Sodium Houttuyfonate (SH, Shanghai Qingping Pharmaceutical Company Ltd., China. Lot No. 100305, Purity 99.4%); Bleomycin (BLM, Haerbin Bolai

Pharmaceutical Company Ltd, Heilongjiang Province, China. Lot No. 100131), Prednisone acetate tablets (PA, Zhejiang Xianju Pharmaceutical Company Ltd. Zhejiang Province, China. Lot No. 100341).

Animal Care and Handling

Kunming mice were obtained from Shanghai Experimental Animal Center, Chinese Academy of Sciences. The animals were housed in stainless steel metabolite cages and observed under a 12 hours light-12 hours dark cycle in a well-ventilated room at 23° C±2° C. They were fed with standard pellet food and tap water ad libitum. The study was approved by the Animal Care and Use Committee of Shanghai University of Traditional Chinese Medicine, and was performed in accordance with the National Institute of Health guidelines for the care and handling of animals.

Administration of Reagents and Study Groups

One hundred and twenty mice (male, 8 weeks old, 18-22 g) were randomly divided into the following 5 groups: sham control operation group (Control); BLM treatment group (BLM); prednisone acetate treatment group (BLM+PA); high concentration SH treatment group (BLM+SH High); and low concentration SH treatment group (BLM+SH Low). In the control group, mice received 1 ml/kg 0.9% sterile saline intratracheally under pentobarbital sodium anesthesia. In the BLM group, mice received a single dose of BLM intratracheally (5 mg in 1 ml sterile saline/body weight (kg)). The control and BLM groups received saline (oral, 5 ml/body weight (kg)) once a day for 4 weeks after BLM injection. We selected the doses of SH based on our previous report and small-scale preliminary experiments. We employed two different administration concentrations. SH was administered orally once a day to both the high (90 mg/kg) and low (45 mg/kg) concentration SH groups. On the 7th, 14th or 28th day, the mice were anesthetized with pentobarbital sodium (50 mg/kg) intraperitoneally.

Histological Examination and Evaluation

The lungs were immersed in buffered formalin (10%) for 48 h, then embedded in paraffin and sliced (5 μm) and stained with H&E, or Sirius red. Each sample slice was photographed (x200 magnification) under the microscope (Olympus BX51, 167 Japan). All photos were analyzed with the image-Pro Plus 6.3 analyzing software (Media Cybernetics, Bethesda, Md. USA) by computer. Type I and type III collagen accumulation in the interstitial space of the lung was assessed by polarized light microscopy. Type I or type III collagen volume fraction (CVF) was also determined with Image-Pro 6.3 analyzing software.

Statistical Analysis

All values were expressed as mean±standard deviation (SD). Statistical analysis was performed by one-way analysis of variance for multiple comparisons, followed by Student-Newman-Keuls test to evaluate the difference between two groups. Values of p<0.05 were considered statistically significant for all analyses.

Results

Effects of SH on lung/body weight ratio in BLM mice

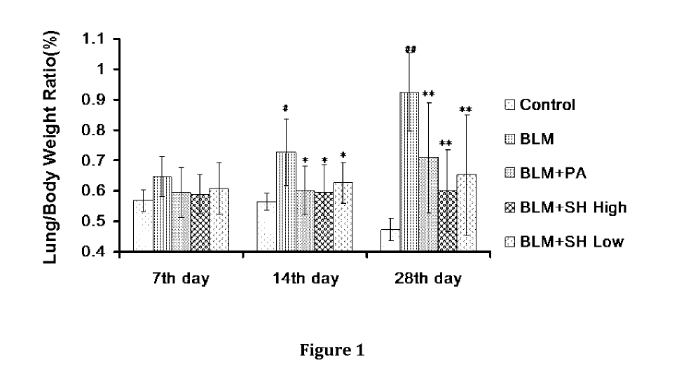

Two different dosages of BLM (2.5 mg/kg, 5mg/kg) were given to 10 mice (5 mice for each group) for small-scale preliminary experiments. On the 7th and 14th day, the mice were anesthetized with pentobarbital sodium (50 mg/kg) intraperitoneally. The lung was collected for histological examination. In the 5 mg/kg treatment group, the lung tissue showed the fibrotic morphology and there is no death in all mice, while the pathological change in the 2.5 mg/kg group was not significant (Data not shown). Therefore, 5 mg of BLM in 2.5 ml sterile saline/body weight (kg) was used to generate BLM induced pulmonary fibrosis mice model. In the BLM group, mice received a single dose of BLM. After the instillation of saline or BLM, the lung/body weight ratio were obtained on the 7th, 14th, and 28th day. As shown in FIG. 1, the lung/body weight increased significantly on the 14th and 28th day mice after BLM treatment. Both PA and SH reduced the lung/body weight ratio on the 14th and 28th day in BLM mice (*p<0.05, **p<0.01). Meanwhile, there was no significant difference in body weight among all groups. This result indicates that higher dose of SH reduces the weight of the lung without adversely affecting the body weight of the tested animals.

Effects of SH on histological changes in BLM mice

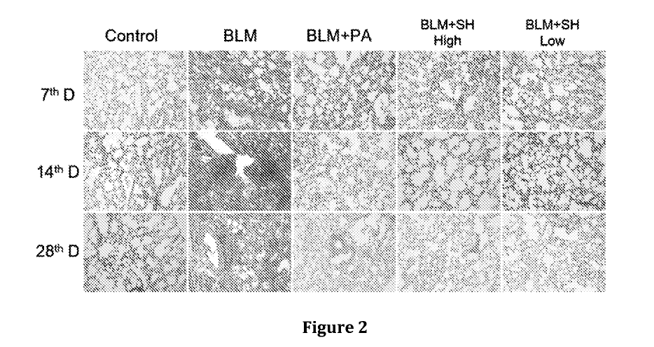

The change in lung/body weight ratio after the administration of BLM suggested that the pulmonary fibrosis mouse model was successfully developed. SH exhibited a protective effect against the BLM induced increase in lung/body weight ratio. We then performed histological examination to investigate the effects of SH on lung morphology in BLM mice. In FIG. 2, it is shown (from left to right): Control group (Control); Bleomycin treated group (BLM); Bleomycin and prednisone acetate treated group (BLM+PA); Bleomycin and sodium houttuyfonate 90 mg/kg treatment group (BLM+SH High); Bleomycin and sodium houttuyfonate 45 mg/kg treatment group (BLM+SH Low). From Top to bottom: Samples were collected at 7th, 14th and 28th day after BLM administration. As shown in FIG. 2, BLM could severely increase lung inflammatory cell infiltration and edema in H&E stained slides in a time dependent manner (FIG. 2 Control and BLM panels). Thickening of the alveolar wall, infiltration of inflammatory cells and fibrosis of alveolar septa were also observed in BLM-treated mice. Fibrotic lesions clearly developed in the interalveolar area of mice lung 14 days after bleomycin treatment and progressed to Day 28.

The PA and SH treatment (both high and low concentrations) reduced the lung inflammatory cell infiltration and collagen deposition (FIG. 2 BLM+PA, BLM+SH High, BLM+SH Low panels). There was no significant difference between PA and SH group. The histological study indicates that SH (both high and low concentrations) is effective in reducing inflammation and collagen deposition in BLM induced pulmonary fibrosis.

Effects of SH on collagen deposition in BLM mice

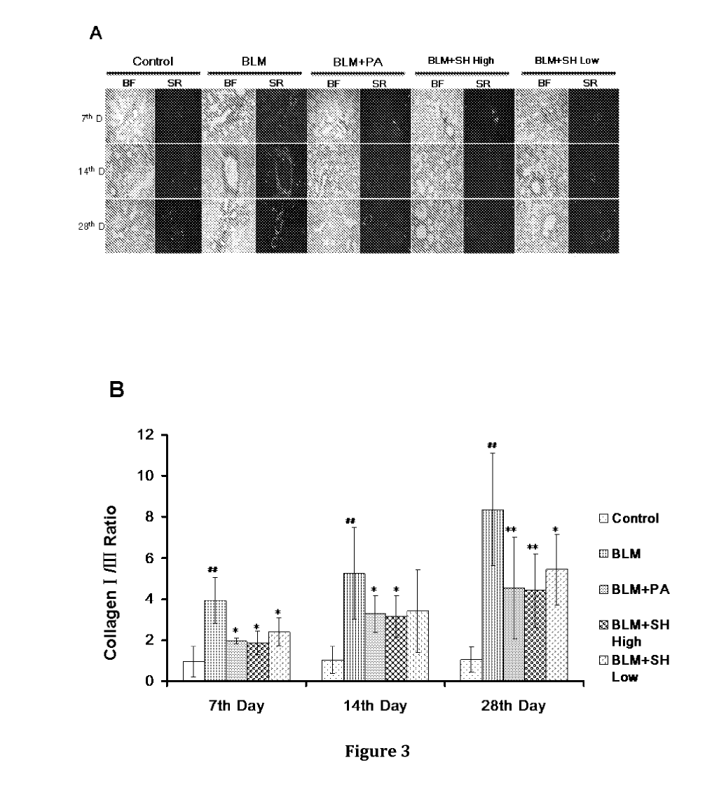

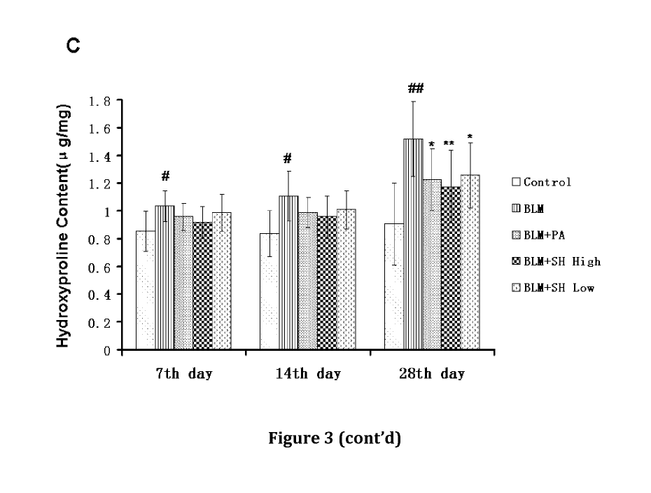

The hallmark characteristic of fibrosis is the excessive deposition of extracellular matrix, such as collagen. Sirius Red staining was employed to examine the pathological changes of collagens. In our experiments, five micrometer-thick sections were stained to identify collagen fibers under polarization microscope. In FIG. 3, it shows how lungs were removed from the animals on the 7th, 14th and 28th day after treatment with saline (Control), BLM alone (BLM), BLM plus prednisone acetate (BLM+PA), BLM plus 90 mg/kg Sodium Houttuyfonate (BLM+SH High), or BLM plus 45 mg/kg Sodium Houttuyfonate (BLM+SH Low) as described in Materials and Methods. Type I collagen was in yellow or orange and type III collagen was green under polarized light. With Sirius red staining and under polarized light observation, increased amounts of type III collagen (green) and type I collagen (yellow) were observed, which were deposited in disoriented fashion in areas of fibrosis (FIG. 3A, BLM group). Lung sections from control animals did not show fibrosis or injury (FIG. 3A, Control group). PA and SH treatment reduced the deposition of collagens in BLM groups in all the sample collection time points (FIG. 3A, BLM+PA, BLM+SH High, BLM+SH Low group). The increase of collagen I/collagen III ratio was one of the markers for pulmonary fibrosis development. To investigate the change of collagen I/III ratio, the collagen contents in various group were analyzed and quantified as statistical data in FIG. 3B. The data showed that BLM treatment increased the collagen I/III ratio from day 7 to day 28. All three drug administration groups (BLM+PH, BLM+SH High, BLM+SH Low) appeared to have low collagen I/III ratios at all time points. Hydroxyproline contents in lung tissue were used as a quantitative index of fibrogenesis and fibrosis. The hydroxyproline contents in the BLM and the BLM plus treatment groups are therefore examined (FIG. 3C). Both PA and SH suppressed the hydroxyproline production in the 28th day samples (*p<0.05, **p<0.01).

The results show that SH is effective in preventing BLM induced pulmonary fibrosis through decreasing collagens production and the collagen I/III ratio. Hydroxyproline content analysis further proves that SH have a similar effect as PA against fibrogenesis and fibrosis.

Discussion

In the present invention, the administration of SH is shown to suppress the increase of lung/body weight ratio, pulmonary collagens contents and hydroxyproline contents in vivo induced by BLM. To examine and confirm the protective properties of SH, two SH treatment groups have been tested in the present invention: the high concentration (90 mg/kg) and low concentration (45 mg/kg) groups. Both concentrations of SH show protective effects against pulmonary fibrosis in all the studied parameters, with no significant differences. These results indicate that SH has reduced BLM toxicity, particularly pulmonary fibrosis, and is a candidate drug for IPF. BLM is known to be a representative inducer of pulmonary fibrosis and is used in experimental models of IPF. BLM has also been clinically used as a chemotherapeutic agent for the treatment of malignant conditions such as squamous cell carcinomas and lymphomas. However, its clinical use is limited by the risk of severe pulmonary toxicity, and there is as yet no established treatment for the prevention of this adverse reaction. The results of the present invention helps solve this problem by preventing pulmonary toxicity in patients administered with BLM.

The role of SH in the regulation of the inflammatory response is of particular interest as the compound not only inhibits pro-inflammatory mediators but actively participates in the resolution of inflammation, preventing an over exuberant inflammatory response and limiting damage to the host. SH has the ability to inhibit cytokine (IL-1β, IL-6 and TNF-α) production in lung tissues of BLM mice, which suggests that SH could also be used as an anti-inflammatory drug in lung infections. Indeed, SH is generally used as an antimicrobial medicine in clinical TCM practice. Despite its long clinical use as an anti-inflammatory agent, the present invention is the first to describe the effects of SH on cytokines expression in BLM mice and provide further experimental evidence on how SH exerts its pharmacological and therapeutic values in the treatment of pulmonary fibrosis in mice.

The hallmark characteristic of fibrosis, namely the excessive deposition of extracellular matrix such as collagen, was markedly inhibited by SH in BLM mice. The presence of myofibroblasts in patients with pulmonary fibrosis is documented in lung tissues as well as animal models of the disease.

In summary, the beneficial effects of SH in the prevention of pulmonary fibrosis induced by BLM are provided in the present invention. The experimental results also indicate that SH is an attractive candidate against IPF and pulmonary toxicity induced by BLM.

### INDUSTRIAL APPLICABILITY

The present invention discloses the use a synthetic compound for its therapeutic uses. More particularly, it relates to compound Sodium Houttuyfonate that is an addition compound of sodium bisulfite and houttuynin, and its biological activity of inhibiting idiopathic pulmonary fibrosis and bleomycin induced pulmonary fibrosis.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A method for treating or preventing pulmonary fibrosis in a subject in need thereof comprising administering to a subject a composition comprising at least one derivative compound from sodium bisulfite and houttuynin or the chemical equivalent thereof.

2. The method according to claim 2 wherein said derivative compound comprises sodium houttuyfonate or the chemical equivalent thereof.

3. The method according to claim 1 wherein said pulmonary fibrosis comprises idiopathic pulmonary fibrosis and bleomycin induced pulmonary fibrosis.

4. The method according to claim 1 wherein said derivative compound is used in preventing or attenuating pulmonary toxicity in said subject with bleomycin induced pulmonary fibrosis.

5. The method according to claim 1 wherein said derivative compound is administered orally to said subject.

6. The method according to claim 5 wherein the oral administration is at a dosage between 45 mg/kg bodily weight and 90 mg/kg bodily weight for at least once a day.

7. The method according to claim 1 wherein said derivative compound is used in preventing pulmonary toxicity in said subject having been administered or administering with bleomycin as chemotherapeutic agent which causes bleomycin induced pulmonary fibrosis.

8. A composition for treating or preventing pulmonary fibrosis in a subject in need thereof comprising at least one derivative compound from sodium bisulfite and houttuynin or the chemical equivalent thereof.

9. The composition according to claim 8 wherein said derivative compound comprises sodium houttuyfonate or the chemical equivalent thereof.

10. The composition according to claim 8 wherein said pulmonary fibrosis comprises idiopathic pulmonary fibrosis and bleomycin induced pulmonary fibrosis.

11. The composition according to claim 8 wherein said derivative compound is used in preventing or attenuating pulmonary toxicity in said subject with bleomycin induced pulmonary fibrosis.

12. The composition according to claim 8 wherein said derivative compound is administered orally to said subject.

13. The composition according to claim 12 wherein the oral administration is at a dosage between 45 mg/kg bodily weight and 90 mg/kg bodily weight for at least once a day.

14. The composition according to claim 8 wherein said derivative compound is used in preventing pulmonary toxicity in said subject having been administered or administering with bleomycin as chemotherapeutic agent which causes bleomycin induced pulmonary fibrosis.
